(19) United States
(12) Patent Application Publication
(10) Pub. No.: US 2014/0163530 A1
(43) Pub. Date: Jun. 12, 2014

(54) NON-PRESSURE TRANSDUCING FLUSHABLE CATHETER

(71) Applicant: Wake Forest University Health Sciences, Winston-Salem, NC (US)

(72) Inventor: Mark B. Frenkel, Winston-Salem, NC (US)

(73) Assignee: Wake Forest University Health Sciences, Winston-Salem, NC (US)

(21) Appl. No.: 14/092,183

(22) Filed: Nov. 27, 2013

Related U.S. Application Data
(60) Provisional application No. 61/732,308, filed on Dec. 1, 2012.

(51) Int. Cl. A61M 25/00; A61M 27/00

(52) U.S. Cl. CPC A61M 25/0028; A61M 27/00
USPC 604/540

(57) ABSTRACT

The present invention provides catheters whose lumen may be cleared of obstructions without transducing pressure changes to the patient compartment in which its proximal end is placed. In an illustrative embodiment, the lumen is cleared by a flush fluid supplied from or to a secondary lumen, with the patient compartment isolated with a gate mechanism. In another illustrative embodiment, the lumen is cleared by a progressively expanding balloon therein.

### RELATED APPLICATIONS

This application claims the benefit of U.S. Provisional Application Ser. No. 61/732,308, filed Dec. 1, 2012, the disclosure of which is incorporated by reference herein in their entirety.

### FIELD OF THE INVENTION

The present invention relates generally to devices and methods for assisting with the flushing or recanalization of a catheter or other device in a fluid or soft tissue space.

### BACKGROUND OF THE INVENTION

Catheters (hollow lumen tubes) are commonly passed through physical tissue structures in order to be inserted into fluid collections, potential spaces, or soft tissue areas in order to allow for drainage, pressure equilibration or monitoring, injection, or other uses of these spaces. These catheters are most commonly single-lumen systems that communicate with a body compartment proximally and the outside environment distally.

These catheters are prone to clogging (obstruction of their lumens) by a number of different materials, including clots and tissue fragments. Often, the catheters must be flushed (fluid flushed into the lumen under pressure) or aspirated (negative pressure applied to the lumen) in order to unclog them. See, e.g., US Patent Application Publication No. US 2005/0171493 to Nicholls.

Common applications of catheter placement include the cases of external ventricular drains (EVDs) or subdural drains (SDDs) where the distal end of the catheter is in communication with the intracranial space. See, e.g., PCT Application WO 2013/086521 to Mourad et al.

Under normal conditions, the intracranial space is comprised of three components: brain parenchyma, cerebrospinal fluid (CSF), and blood. Increased pressure in this compartment may lead to herniation of the brain out of its normal anatomical location and serious consequences. To avoid this, EVDs and SDDs are commonly used to drain blood or CSF and to monitor pressure in the intracranial compartment.

In the cases of catheter clogging in EVDs and SDDs, aspiration and flushing are suboptimal solutions as any pressure (either positive or negative) applied to the lumen of the catheter has the potential to be transduced into the intracranial space and cause complications by either increasing or decreasing intracranial pressure (ICP), or even causing herniation of brain tissue into the catheter lumen in the case of aspiration.

As a result, clogged EVD and SDD catheters commonly require replacement, which often requires retunneling of the catheter through brain tissue (in the case of the former) and may lead to severe complications.

### SUMMARY OF THE INVENTION

The present invention seeks to provide devices and methods for a catheter system whose lumen may be cleared of any obstructions without transducing pressure changes to the patient or subject compartment in which its proximal end is placed.

Accordingly, disclosed herein is a flushable drain catheter, comprising:

(i) an elongate catheter body having a distal end portion a proximal end portion, a side wall portion, a primary lumen therein, and a secondary lumen therein, with the primary lumen and the secondary lumen separated by a septum;

(ii) at least one exterior drain opening formed in the catheter body side wall adjacent the proximal end portion;

(iii) at least one internal flush opening formed in the septum at or adjacent the proximal end portion; and

(iv) a gate operatively associated with the at least one exterior drain opening, the gate switchable between a drain configuration in which the at least one drain opening is open, and a flush configuration in which the at least one drain opening is closed.

Also disclosed herein is a flushable drain catheter, comprising:

(i) an elongate catheter body having a distal end portion, an intermediate portion, and a proximal end portion, a side wall portion and a primary lumen formed therein;

(ii) at least one exterior drain opening formed in the catheter body side wall in or adjacent the proximal end portion;

(iii) an elongate balloon element positioned in the primary lumen and extending from the proximal end portion to at least the intermediate portion, the balloon having a distal end portion, an intermediate portion, and a proximal end portion. The balloon element is configured to be progressively inflatable, from proximal to distal, from a fully deflated position in which both the drain opening and the primary lumen are open, through a partially inflated position in which the drain opening is closed and the lumen intermediate portion is open, to a fully inflated position in which the drain opening, the lumen proximal portion, and at least the lumen intermediate portion are closed. Thus, inflation of the balloon element forms a balloon face portion that progressively advances from proximal to distal in the lumen that flushes or ejects debris therefrom.

The present invention has many applications, and may be used or employed with any indwelling drainage catheter, examples of which include but are not limited to external ventricular drains and subdural drains, and the catheter body may be rigid or flexible depending upon the particular purpose for which the device is employed.

The foregoing and other objects and aspects of the present invention are explained in greater detail in the drawings herein and the specification set forth below. The disclosures of all United States patents references cited herein are to be incorporated herein by reference in their entirety.

### BRIEF DESCRIPTION OF THE DRAWINGS

FIG. 1A is a schematic illustration of a first embodiment of a catheter of the present invention.

FIG. 1B is a schematic illustration of the embodiment of FIG. 1A showing the opposite side thereof.

FIG. 1C is a side-sectional schematic illustration of the embodiment of FIG. 1A-1B, showing the rotating gate, where the catheter is in the “drain” configuration.

FIG. 1D is a side-sectional schematic illustration of the embodiment of FIGS. 1A-1C, except that the catheter is now in the “flush” configuration.

FIG. 2A is a cross-sectional schematic view of a second embodiment of the invention, where the catheter is in the “drain” configuration.

FIG. 2B is a cross-sectional schematic view of the embodiment of FIG. 2A, except that the catheter is now in the “flush” configuration.

FIG. 3A is a side-sectional schematic illustration of a third embodiment of the present invention employing a sliding gate, where the catheter is in the “drain” configuration.

FIG. 3B is a side-sectional schematic illustration of the embodiment of FIG. 3A, except that the catheter is now in the “flush” configuration.

FIG. 4A is a side-sectional schematic illustration of a fourth embodiment of the present invention employing an inflatable gate, where the catheter is in the “drain” configuration.

FIG. 4B is a side-sectional schematic illustration of the embodiment of FIG. 4A, except that the catheter is now in the “flush” configuration.

FIG. 5A is a side-sectional schematic illustration of a single-lumen embodiment of the present invention, where the catheter is in the “drain” configuration.

FIG. 5B is a side-sectional schematic illustration of the embodiment of FIG. 5A, with the balloon partially inflated, and transitioning from the “drain” to “flush” configuration.

FIG. 5C is a side-sectional schematic illustration of the embodiment of FIGS. 4A-4B, fully transitioned to the “flush” configuration, during an early phase of debris ejection.

FIG. 5D is a side-sectional schematic illustration of the embodiment of FIGS. 5A-5C, fully transitioned to the “flush” configuration and during a late phase of debris ejection.

### DETAILED DESCRIPTION OF ILLUSTRATIVE EMBODIMENTS

The present invention now will be described more fully hereinafter with reference to the accompanying figures, in which embodiments of the invention are shown. This invention may, however, be embodied in many alternate forms and should not be construed as limited to the embodiments set forth herein. Like numbers refer to like elements throughout the description of the figures.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety.

Also as used herein, “and/or” refers to and encompasses any and all possible combinations of one or more of the associated listed items, as well as the lack of combinations when interpreted in the alternative (“or”).

Catheters as described herein may be made from materials known in the art (see, e.g., U.S. Pat. Nos. 4,692,141 and 5,653,684; US Patent Application Pub. No. US 2008/0294145) or variations thereof that will be apparent based on the examples given herein. For example, balloons or balloon elements may be made of any suitable elastic polymer material. The catheters can include or be made from radio-opaque or radiodense materials so that they can be seen on computer-assisted tomography (CT), ultrasound-guided placement, or the like, as is known in the art.

### 1. Dual Lumen Flushable Catheters.

The present invention can be embodied in a variety of different physical forms.

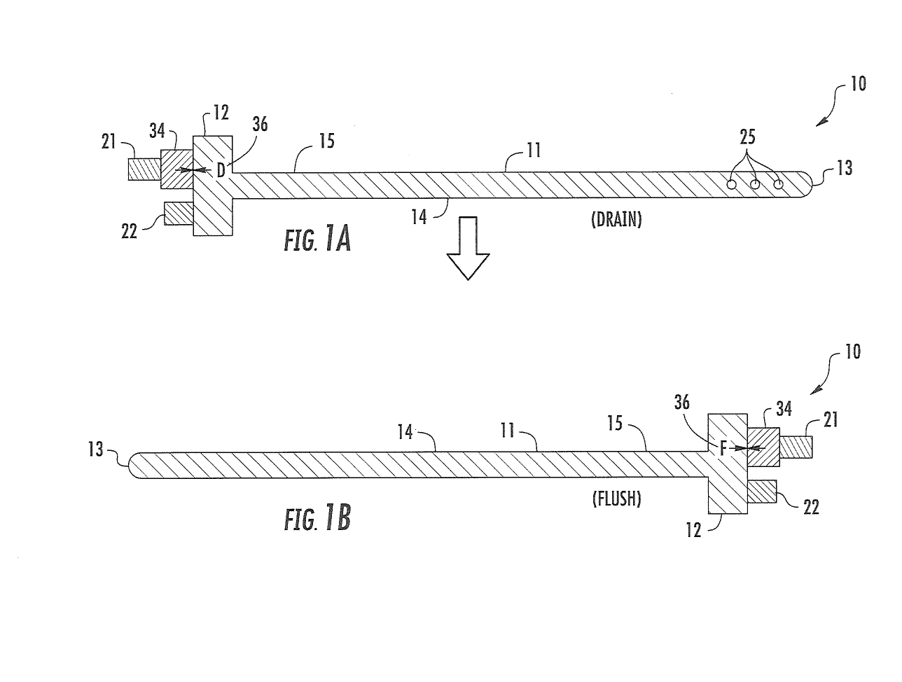

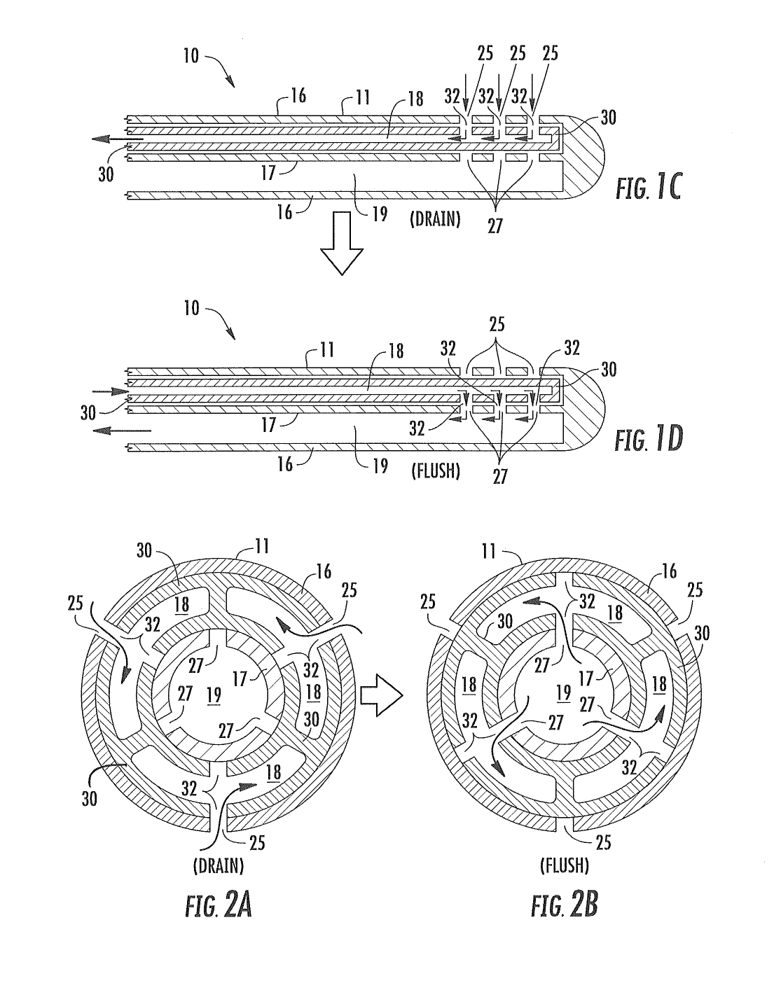

A first non-limiting example is given in FIGS. 1A-1D, which shows a drain catheter 10 that has an elongate catheter body 11 connected to a distal (or external) hub 12. The catheter body generally comprises a proximal (with respect to the patient) end portion 13, an intermediate portion 14, and a distal portion 15. The catheter body includes a side wall portion 16, and also includes an internal wall or septum 17 dividing, and defining, a primary lumen 18 and a secondary lumen 19. A primary external port 21 (such as a Luer lock syringe fitting) can be provided in fluid communication with the primary lumen, and a secondary external port 22 (again, such as a Luer lock syringe fitting) can be provided in fluid communication with the secondary lumen. The primary and secondary lumens may be adjacent to or concentric with one another, as further illustrated below, and may be of any suitable cross-sectional shape, including circular, polygonal, D-shaped, etc.

The catheter body has at least one, and generally a plurality, of exterior drain openings 25 formed in the catheter body adjacent the proximal end portion (with the openings optionally extending further up along the catheter body, up to the intermediate portion, and with the openings formed on multiple sides of the catheter body (not visible in the Figures). The drain opening(s) may be any suitable shape, size, or geometric arrangement on the catheter body. In addition, the catheter body has at least one, and in some embodiments a plurality, of internal flush openings 27 formed in the septum, again at or adjacent the catheter body proximal end portion.

A gate 30 is provided in the catheter body, in operable association with the at least one exterior drain opening, which gate is switchable between a drain configuration (FIG. 1A and FIG. 1C, and like-labeled Figures discussed below) and a flush configuration (FIG. 1B and FIG. 1D, and like-labeled Figures discussed below). In the drain configuration, the at least one drain opening 25 is open, and in the flush configuration the at least one drain opening 25 is closed. Where multiple or a plurality of drain openings 25 are present, some, most, or all are open in the drain configuration, and all are closed in the flush configuration. The flow of biological fluid, from within the patient or subject (e.g., from a ventricle, the subdural space, peritoneal cavity, an abscess, etc.) through the drain openings and primary lumen and (ultimately) out the primary port, while the catheter is in the drain mode is illustrated by arrows). Likewise, the flow of flush fluid, from the secondary lumen through the flush openings into the primary lumen (or vice-versa) when the catheter is in the flush configuration, is illustrated by arrows).

As discussed below, the gate may take any of a variety of physical forms. In the embodiment of FIGS. 1A-1D, the gate comprises a rotating gate 30 member having a gate port, or a plurality of gate ports 32 formed therein. The port or ports may be of any suitable shape, size, or pattern. The same gate ports that cooperate with the drain openings may also cooperate with the flush openings (as illustrated in FIGS. 1C-1D, or different gate ports may be provided for cooperation with the flush openings. Whatever specific arrangement, the gate port or ports are readily configured so that the drain openings must be fully closed before the flush openings are open, thereby aiding in the prevention of inadvertent introduction of flush fluid into the patient.

The rotating gate 30 shown in FIGS. 1C-1D is operatively connected to with an external gate control 34 (shown in FIGS. 1A-1B) which, when rotated, switches the gate from the drain configuration to the flush configuration. An external indicator in the form of a printed and/or tactile indicia 36, specifically in the form of arrows, a “D” for “drain” and an “F” for “flush,” are printed and/or otherwise formed on the hub and gate control to provide an external visual indication of the status of the gate and the configuration of the catheter in drain or flush mode, though numerous other indicators can be employed, including different visual and/or tactile indicia, and other mechanical and/or electromechanical indicia such as lights, sounds, etc.

As noted above, the gate element may take a variety of different physical forms. In the additional examples illustrating some of these forms below, like elements are assigned like numbers throughout. Those skilled in the art will appreciate still other gate arrangements that will be apparent from these non-limiting examples.

FIGS. 2A-2B provide another example of a rotating gate element. Here the secondary lumen 19 is positioned concentrically within the primary lumen 18, and the gate element rotates around the septum 17. The gate element itself comprises an inner and outer wall that extend continuously throughout the primary lumen and contain the flow of fluid therein, which inner and outer wall are joined for rotation by intermittently spaced pillars that permit the flow of fluid therearound. Again note that the gate ports are configured (and can be re-arranged and further configured) so that the drain openings must be fully closed before the flush openings are open, thereby aiding in the prevention of inadvertent introduction of flush fluid into the patient.

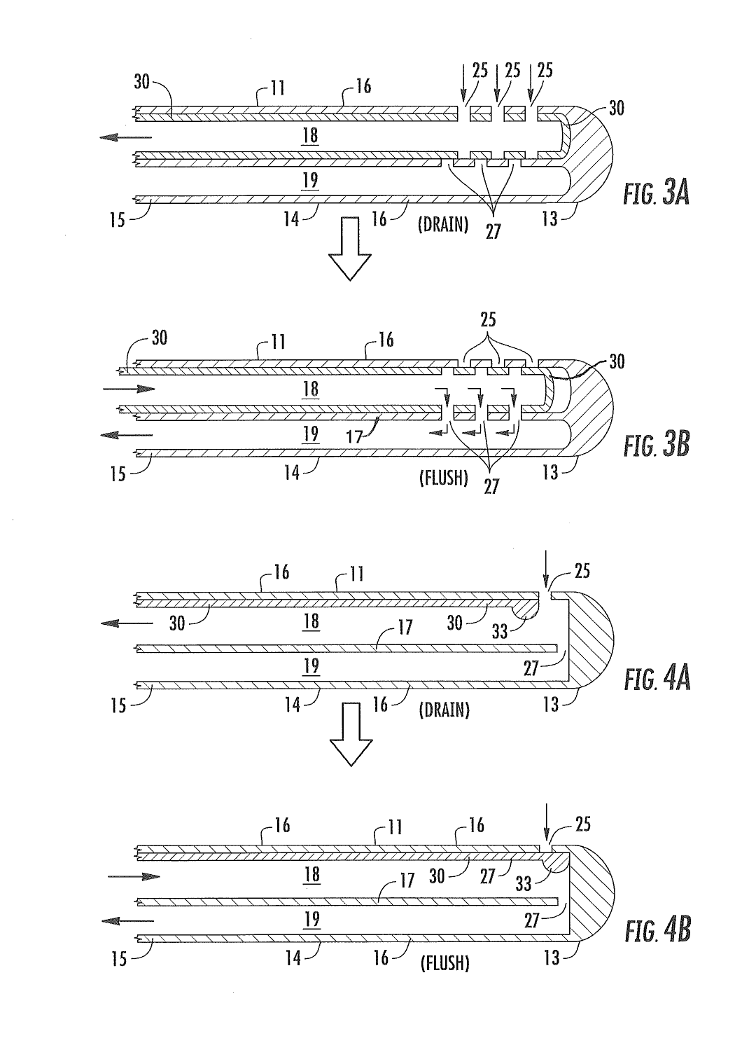

FIGS. 3A-3B illustrate a sliding gate element, where different gate ports are arranged to cooperate with the drain openings and the gate openings. Although not illustrate for clarity in the schematic drawings provided, note that spacing and/or geometry of the gate ports may be easily arranged so that, which switching the catheter from the drain configuration to flush configuration, all of the drain openings must be closed, before any of the flush openings are open, thereby aiding in the prevention of inadvertent introduction of flush fluid into the patient.

FIG. 4A-4B illustrate an inflatable gate member, in which a proximal balloon is positioned to close the drain openings, which proximal balloon 33 can be expanded by introduction of an inflation fluid (air, saline, etc). Again, the balloon gate element may be configured so that the flush opening is only open when the drain element is fully closed, for example, by the provision of a second balloon operatively associated with the flush opening, with the first and second balloon in fluid communication with one another, and inflation of the one accomplished by pumping (by an external pump) of fluid from, and concurrent deflation of, the other, during switching from the drain configuration to the flush configuration (which process may be reversed to switch back to the drain configuration).

As an alternative to an inflatable balloon catheter as a gate element, an expandable wire mesh element (not shown) may be used, where pushing forward or pulling back on a wire reaching from the distal tip back through the catheter body expands a wire network or mesh (optionally having an overlying elastic sleeve or balloon) and mechanically occludes the at least one drain opening. Numerous examples of such wire mesh elements are known, including but not limited to that set forth in U.S. Pat. No. 5,653,684 to Laptewicz et al., the disclosure of which is incorporated by reference herein in its entirety.

In any of the embodiments described above, a retractable brush element (see, e.g, US Patent Application Publication No. US 2005/0171493), a retractable expanding wire mesh element, and/or retractable inflatable balloon catheter element, may be placed within either or both lumens, to further provide a mechanical means for ejecting debris or solid material from within the catheter.

### 2. Single Lumen Flushable Catheters.

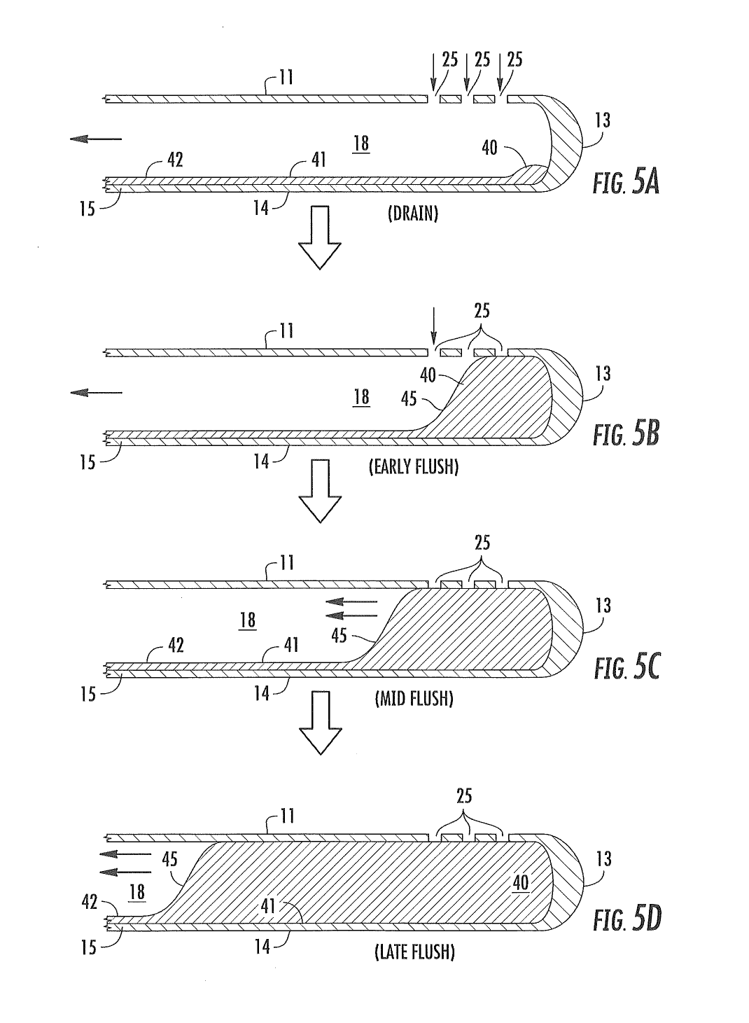

FIGS. 5A-5D illustrate an alternate embodiment of the invention, in which a single lumen is used and flushing is achieved mechanically. The device again has an elongate catheter body 11 which is connected to an external or distal hub (not shown). The catheter body generally comprises a proximal (with respect to the patient or subject) end portion 13, an intermediate portion 14, and a distal portion 15. The catheter body includes a side wall portion 16 as in the embodiments above, and at least one at least one exterior drain opening 25 formed in the catheter body side wall at or adjacent the proximal end portion, as also described above.

In this embodiment, an elongate balloon element 33′ is positioned in the primary lumen, extending from the proximal end portion to at least the intermediate portion and optionally on up to the distal portion. The balloon element may be fixed in the lumen, or may be retractable from the lumen when in its deflated position. The balloon element itself has a proximal end portion 40, an intermediate portion, 41, and optionally (but in some embodiments preferably) a distal portion 42, which are configured to be progressively and continuously inflatable, from proximal to distal. This progressive inflation, which causes the balloon to progressively and completely fill the lumen, forms a balloon face portion 45 which acts as a “plunger” that progressively advances from proximal to distal in the lumen and flushes or ejects debris therefrom. Expansion of the balloon in the lumen substantially equalizes pressure during the flush process, by progressively closing off the drain openings, so that pressure changes to which the subject or patient may be exposed to during the flush process are minimized.

The foregoing is illustrative of the present invention, and is not to be construed as limiting thereof. The invention is defined by the following claims, with equivalents of the claims to be included therein.

That which is claimed is:

1. A flushable drain catheter, comprising:
an elongate catheter body having a distal end portion, a proximal end portion, a side wall portion, a primary lumen therein, and a secondary lumen therein, with said primary lumen and said secondary lumen separated by a septum;
at least one exterior drain opening formed in said catheter body side wall adjacent said proximal end portion;
at least one internal flush opening formed in said septum at or adjacent said proximal end portion; and
a gate operatively associated with said at least one exterior drain opening, said gate switchable between a drain configuration in which said at least one drain opening is open, and a flush configuration in which said at least one drain opening is closed.

2. The flushable drain catheter of claim 1, wherein said gate comprises a rotating gate member.

3. The flushable drain catheter of claim 1, wherein said gate comprises a sliding gate member.

4. The flushable drain catheter of claim 1, wherein said gate comprises an inflatable gate member.

5. The flushable drain catheter of claim 1, wherein said gate comprises an expandable wire gate member.

6. The flushable drain catheter of claim 1, wherein said catheter body is rigid.

7. The flushable drain catheter of claim 1, wherein said catheter body is flexible.

8. The flushable drain catheter of claim 1, wherein said gate is configured so that said at least one internal flush opening is closed when said gate is in said drain configuration, and said at least one internal flush opening is open when said gate is in said flush configuration.

9. The flushable drain catheter of claim 1, wherein said gate is configured so that all of said at least one internal flush opening(s) is or are only open when all of said at least one exterior drain opening(s) is or are fully closed.

10. The flushable drain catheter of claim 1, wherein said catheter further comprises an external indicator operatively associated with said gate, said indicator configured to identify whether said catheter is in said flush configuration or said drain configuration.

11. The flushable drain catheter of claim 1, wherein said catheter is an exterior ventricular drain or subdural drain

12. A flushable drain catheter, comprising:
an elongate catheter body having a distal portion, an intermediate portion, a proximal end portion, a side wall and a primary lumen therein;
at least one exterior drain opening formed in said catheter body side wall adjacent said proximal end portion;
an elongate balloon element positioned in said primary lumen and extending from said proximal end portion to at least said intermediate portion, and optionally to said distal portion, said balloon having a proximal end portion, an intermediate portion, and optionally a distal end portion,
said balloon element configured to be progressively inflatable, from proximal to distal, from a fully deflated position in which both said drain opening and said lumen are open, through a partially inflated position in which said drain opening is closed and said lumen intermediate portion is open, to a fully inflated position in which said drain opening, said lumen proximal portion, and at least said lumen intermediate portion are closed,
with inflation of said balloon element forming a balloon face portion that progressively advances from proximal to distal in said lumen that flushes or ejects debris therefrom.

13. The drain catheter of claim 10, wherein said elongate balloon element is fixed in said lumen.

14. The drain catheter of claim 10, wherein said elongate balloon element is retractable from said lumen when in said deflated position.

15. The flushable drain catheter of claim 10, wherein said catheter body is rigid.

16. The flushable drain catheter of claim 10, wherein said catheter body is flexible.

17. The flushable drain catheter of claim 10, wherein said catheter is an exterior ventricular drain or subdural drain.
